(19) United States
(12) Patent Application Publication
(10) Pub. No.: US 2008/0214595 A1
(43) Pub. Date: Sep. 4, 2008

(54) Use Of Rapamycin Derivatives For The Treatment And/Or Prevention Of Cardiovas Cular Disorders

(76) Inventors: Seigo Izumo, Brookline, MA (US); Stefan Edwin Hardt, Heidelberg (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/914,314

(22) PCT Filed: May 15, 2006
(86) PCT No.: PCT/US06/18656

§ 371 Date: Nov. 13, 2007

Related U.S. Application Data
(60) Provisional application No. 60/681,550, filed on May 16, 2005.
(60) Provisional application No. 60/681,654, filed on May 17, 2005.

(51) Int. Cl. A61K 31/436; A61P 9/00; A61P 9/12; A61P 9/10

(52) U.S. Cl. 514/291

(57) ABSTRACT

The present invention provides methods of treating and/or preventing cardiovascular disorders comprising administering rapamycin derivative compounds.

The present invention relates to the use of rapamycin derivatives for the treatment and/or prevention of cardiovascular disorders, particularly heart failure.

Rapamycin is a known macrolide antibiotic produced by Streptomvces hyngroscopicus having the structure depicted in Formula I:

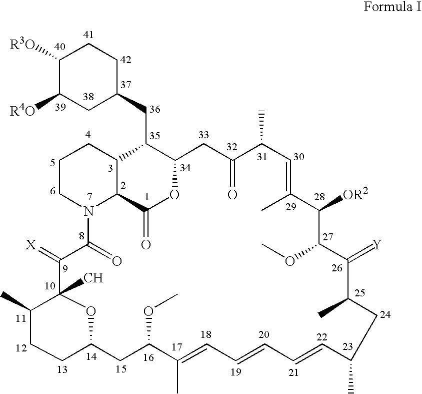

See. e.g. McAlpine. J. B., et al., J. Antibiotics (1991) 44: 688; Schreiber. S. L., et al., J. Am. Chem. Soc. (1991) 113: 7433; U.S. Pat. No. 3,929,992. Rapamycin is an extremely potent immunosuppressant and has also been shown to have antitumor and antifungal activity. Its utility as a pharmaceutical, however, is restricted by its very low and variable bioavailability as well as its high toxicity. Moreover, rapamycin is highly insoluble, making it difficult to formulate stable galenic compositions. Since cardiovascular disease is one of the most prevalent diseases, there exists a strong need for active agents that are suitable for treating and/or preventing cardiovascular diseases.

In accordance with this need, the present invention relates to a new use, in particular a new use for a compound group comprising derivatives of rapamycin, in free form or in pharmaceutically acceptable sail or complex form. Suitable derivatives of rapamycin include e.g. compounds of formula I

wherein

X is (H,H) or O;

Y is (H,OH) or O;

R¹ and R² are independently selected from

H, alkyl, arylalkyl, hydroxyalkyl, dihydroxyalkyl, hydroxyalkoxycarbonylalkyl, hydroxyalkylarylalkyl, dihydroxyalkylarylalkyl, acyloxyalkyl, aminoalkyl, alkylaminoalkyl, alkoxycarbonylaminoalkyl, acylaminoalkyl, arylsulfonamidoalkyl, allyl, dihydroxyalkylallyl, dioxolanylallyl, dialkyl-dioxolanylalkyl, di(alkoxycarbonyl)-triazolyl-alkyl and hydroxy-alkoxy-alkyl; wherein “alk-” or “alkyl” is C₁₋₆ alkyl, branched or linear; “aryl” is phenyl or tolyl; and acyl is a radical derived from a carboxylic acid; and

R⁴ is methyl or

R⁴ and R¹ together form C₂₋₆ alkyl;

provided that R¹ and R² are not both H; and hydroxyalkoxyalkyl is other than hydroxyalkoxymethyl.

Such compounds are disclosed in U.S. Pat. No. 5,665,772 issued Sep. 9, 1997, the contents of which, in particular with respect to the compounds, are incorporated herein by reference as if set forth in full herein.

Acyl as may be present in R₁ or R₂, is preferably Rₐ CO— wherein Rₐ is C₁₋₆ alkyl, C₂₋₆ alkenyl, C₃₋₆ cycloalkyl, aryl, aryl C₁₋₆ alkyl (wherein aryl is as defined above) or heteroaryl, e.g. a residue derived from a 5 or 6 membered heterocycle comprising N, S or O as a heteroatom and optionally one or two N as further heteroatoms. Suitable heteroaryl include e.g. pyridyl, morpholino, piperazinyl and imidazolyl.

Examples of such compounds include:
1. 40-O-Benzyl-rapamycin
2. 40-O-(4′-Hydroxymethyl)benzyl-rapamycin
3. 40-O-[4′-(1,2-Dihydroxyethyl)]benzyl-rapamycin
4. 40-O-Allyl-rapamycin
5. 40-O-[3′-(2,2-Dimethyl-1,3-dioxolan-4(S)-yl)-prop-2′-en-1′-yl]-rapamycin
6. (2′E,4′S)-40-O-(4′,5′-Dihydroxypent-2′-en-1′-yl)-rapamycin
7. 40-O-(2-Hydroxy)ethoxycarbonylmethyl-rapamycin
8. 40-O-(2-Hydroxy)ethyl-rapamycin
9. 40-O-(3-Hydroxy)propyl-rapamycin
10. 40-O-(6-Hydroxy)hexyl-rapamycin
11. 40-O-[2-(2-Hydroxy)ethoxy]ethyl-rapamycin
12. 40-O-[(3S)-2,2-Dimethyldioxolan-3-yl]methyl-rapamycin
13. 40-O-[(2S)-2,3-Dihydroxyprop-1-yl]-rapamycin
14. 40-O-(2-Acetoxy)ethyl-rapamycin
15. 40-O-(2-Nicotinoyloxy)ethyl-rapamycin
16. 40-O-[2-(N-Morpholino)acetoxy]ethyl-rapamycin
17. 40-O-(2-N-Imidazolylacetoxy)ethyl-rapamycin
18. 40-O-[2-(N-Methyl-N′-piperazinyl)acetoxy]ethyl-rapamycin
19. 39-O-Desmethyl-39,40-O,O-ethylene-rapamycin
20. (26R)-26-Dihydro-40-O-(2-hydroxy)ethyl-rapamycin
21. 28-O-Methyl-rapamycin
22. 40-O-(2-Aminoethyl)-rapamycin
23. 40-O-(2-Acetaminoethyl)-rapamycin
24. 40-O-(2-Nicotinamidoethyl)-rapamycin
25. 40-O-(2-(N-Methyl-imidazo-2′-ylcarboxamido)ethyl)-rapamycin
26. 40-O-(2-Ethoxycarbonylaminoethyl)-rapamycin
27. 40-O-(2-Tolylsulfonamidoethyl)-rapamycin
28. 40-O-[2-(4′,5′-Dicarboethoxy-1′,2′,3′-triazol-1′-yl)-ethyl]-rapamycin

A preferred compound is e.g. 40-O-(2-hydroxy)ethyl-rapamycin (referred thereafter as Compound A).

Compounds of formula I have, on the basis of observed activity, e.g. binding to macrophilin-12 (also known as FK-506 binding protein or FKBP-12), e.g. as described in U.S. Pat. No. 5,665,772, been found to be useful e.g. as immunosuppressants, e.g. in the treatment of acute allograft rejection.

Furthermore, suitable derivatives of rapamycin include e.g. compounds of formula II as disclosed in U.S. Pat. Nos. 5,352,671 and 5,912,238, the contents of which, in particular with respect to the compounds, are incorporated herein by reference. The compounds of formula II are represented by the following generic structure:

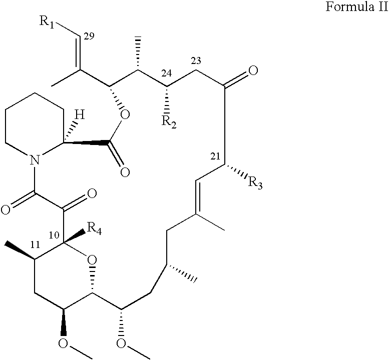

wherein either R₁ is a group (a) of formula:

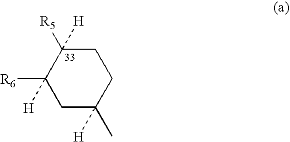

wherein R₅ is chloro, bromo, iodo or azido and R₆ is hydroxy or methoxy;

R₂ is oxo and there is a single bond in 23, 24 position; optionally protected hydroxy and there is a single or a double bond in 23, 24 position; or absent and there is a double bond in 23, 24 position; and

R₄ is hydroxy and there is a single bond in 10, 11 position; or absent and there is double bond in 10, 11 position;

or R₁ is a group (b) or (d) of formula

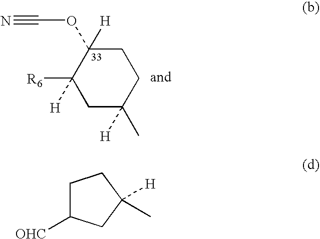

wherein R₆ is as defined above;

R₂ is as defined above; and

R₄ is hydroxy and there is a single bond in 10, 11 position;

Or R₁ is a group (c) of formula:

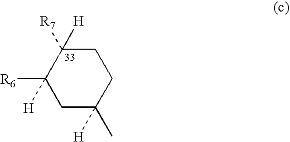

wherein R₆ is as defined above and

R₇ is oxo; optionally protected hydroxy; methoxy; methylthiomethoxy; isobutanoyloxy; aminooxalyloxy; R₈ R₉ CHCOO— wherein R₈ is optionally protected hydroxy or optionally protected amino and R₉ is hydrogen or methyl; or p-tolyloxythiocarbonyloxy;

R₂ is oxo and there is a single bond in 23, 24 position; absent and there is a double bond in 23, 24 position; or is optionally protected hydroxy, methoxy, methylthiomethoxy, isobutanoyloxy, aminooxalyloxy or R₈ R₉ CHCOO— wherein R₈ and R₉ are as defined above, and there is a single or a double bond in 23, 24 position; whereby for group (c),1) when R₇ is oxo, unprotected hydroxy or methoxy then R₂ is other than absent and other than unprotected hydroxy or methoxy, and there is a single bond in 23, 24 position; 2) when R₆ is methoxy and R₇ is methylthiomethoxy then R₂ is other than absent and other than unprotected hydroxy; 3) when R₆ is methoxy and R₇ is protected hydroxy then R₂ is other than optionally protected hydroxy; and 4) when R₆ is hydroxy then R₇ is other than optionally protected hydroxy; and R₄ is hydroxy and there is a single bond in 10, 11 position; and R₃ is methyl, ethyl, n-propyl or allyl; in free form and, where such forms exist, in salt form, hereinafter referred to as “the compounds of the invention”.

As is evident from formula II and the definition of the substituents when there is a single bond in 10, 11 position the carbon atom to which the methyl group in 11 position is attached has the .beta.-configuration and there is a hydrogen atom with the .alpha.-configuration attached to the carbon atom in 11 position; when there is a double bond in 10, 11 position this methyl group lies in the plane of the paper and there is no hydrogen atom in 11 position. When R₂ is oxo no hydrogen atom is attached to the carbon atom in 24 position. When R₇ is oxo the hydrogen atom shown in group (c) attached to the same carbon atom as R₇ is absent.

R₁ preferably is a group (c) or (d). R₂ preferably is unprotected hydroxy and there is a single bond in 23, 24 position. R₃ preferably is ethyl or allyl. R₄ preferably is hydroxy. R₅ preferably is chloro. R₆ preferably is methoxy. R₇ preferably is isobutanoyloxy, aminooxalyloxy or R₈ R₉ CHCOO—. R₈ preferably is unprotected hydroxy or unprotected amino, especially unprotected hydroxy. R₉ preferably is hydrogen. When R₉ is other than hydrogen the carbon atom to which it is attached preferably has the (S) configuration.

Protected hydroxy preferably is hydroxy protected by a conventional hydroxy-protecting group such as formyl, tert-butoxycarbonyl, or trialkylsilyl; it especially is tert-butyldimethylsilyloxy.

Optionally protected hydroxy as defined above under formula I for R₂ and R₇ should not be understood as including a group R₂ or R₇ which is otherwise specified, such as e.g. aminooxalyloxy or R₈ R₉ CHCOO—.

Protected amino preferably is amino protected by a conventional amino-protecting group such as benzyloxycarbonyl or trialkylsilyl; it especially is tert-butoxycarbonyl.

A compound of the invention preferably is in free form. It preferably is in unprotected form.

A subgroup of compounds of the invention is the compounds Ip₁, i.e. the compounds of formula II wherein R₁ is a group (a) wherein R₆ is methoxy and either R₅ is chloro or bromo and R₄ is hydroxy and there is a single bond in 10, 11 position or R₅ is azido and R₄ is hydroxy and there is a single bond in 10, 11 position or absent and there is a double bond in 10, 11 position; R₂ is optionally protected hydroxy and there is a single or a double bond in 23, 24 position; and R₃ is as defined above under formula II; in free form and, where such forms exist, in salt form.

A further subgroup of compounds of the invention is the compounds Ip₂, i.e. the compounds of formula II wherein R₁ is a group (c) wherein R₆ is methoxy and R₇ is oxo; optionally protected hydroxy; methoxy; methylthiomethoxy; aminooxalyloxy; R₈ CH₂COO— wherein R₈ is optionally protected amino; or p-tolyloxythiocarbonyloxy; R₂ is absent and there is a double bond in 23, 24 position; or optionally protected hydroxy, methoxy, methylthiomethoxy or aminooxalyloxy and there is a single or double bond in 23, 24 position; whereby 1) when R₇ is oxo, unprotected hydroxy or methoxy then R₂ is other than absent and other than unprotected hydroxy or methoxy, and there is a single bond in 23, 24 position; 2) when R₇ is methylthiomethoxy then R₂ is other than absent and other than unprotected hydroxy; and 3) when R₇ is protected hydroxy then R₂ is other than optionally protected hydroxy; and R₄ is hydroxy and there is a single bond in 10, 11 position; and R₃ is as defined above under formula II; in free form and, where such forms exist, in salt form.

A further subgroup of compounds of the invention is the compounds Ip₃, i.e. the compounds of formula II wherein R₁ is a group (b) wherein R₆ is methoxy, R₂ is optionally protected hydroxy and there is a single bond in 23, 24 position; or absent and there is a double bond in 23, 24 position; R₄ is hydroxy and there is a single bond in 10, 11 position; and R₃ is as defined above under formula II; in free form and, where such forms exist, in salt form.

A further subgroup of compounds of the invention is the compounds Ip₄, i.e. the compounds of formula II wherein R₁ is a group (d), R₂ is optionally protected hydroxy and there is a single bond in 23, 24 position; or absent and there is a double bond in 23, 24 position; R₄ is hydroxy and there is a single bond in 10, 11 position; and R₃ is as defined above under formula II in free form and, where such forms exist, in salt form.

A preferred subgroup of compounds of the invention is the compounds of formula II wherein R₁ is a group (a) wherein R₅ is as defined above under formula 11 and R₆ is methoxy; R₂ is optionally protected hydroxy and there is a single bond in 23, 24 position; R₄ is hydroxy and there is a single bond in 23, 24 position; or absent and there is a double bond in 10, 11 position; and R₃ is ethyl or allyl.

A further preferred group of compounds of the invention is the compounds of formula II wherein R, is a group (b) wherein R₆ is methoxy; R₂ is optionally protected hydroxy and there is a single bond in 23, 24 position; or absent and there is a double bond in 23, 24 position; R₄ is hydroxy and there is a single bond in 10, 11 position; and R₃ is ethyl or allyl.

A further preferred group of compounds of the invention is the compounds of formula II wherein R₁ is a group (c) wherein R₆ is methoxy and R₇ is as defined above under formula II; R₂ is oxo and there is a single bond in 23, 24 position; or optionally protected hydroxy, methylthiomethoxy, aminooxalyloxy, R₈ CH₂COO— wherein R₈ is optionally protected amino, and there is a single or a double bond in 23, 24 position; whereby 1) when R₇ is oxo, unprotected hydroxy or methoxy then R₂ is other than unprotected hydroxy or methoxy, and there is a single bond in 23, 24 position; 2) when R₇ is methylthiomethoxy then R₂ is other than unprotected hydroxy; and 3) when R₇ is protected hydroxy then R₂ is other than optionally protected hydroxy; R₄ is hydroxy and there is a single bond in 10, 11 position; and R₃ is ethyl or allyl.

A further preferred subgroup of compounds of the invention is the compounds of formula II wherein R₁ is a group (d), R₂ is optionally protected hydroxy and there is a single bond in 23, 24 position; or absent and there is a double bond in 23, 24 position; R₄ is hydroxy and there is a single bond in 10, 11 position; and R₃ is ethyl or allyl.

Especially preferred compounds include compounds disclosed in Examples 1 through 71 of U.S. Pat. No. 5,912,238.

Furthermore, suitable derivatives of rapamycin include e.g. compounds of formula III as disclosed in U.S. Pat. No. 5,985,890, the contents of which, in particular with respect to the compounds, are incorporated herein by reference. The compounds of formula III are represented by the following generic structure:

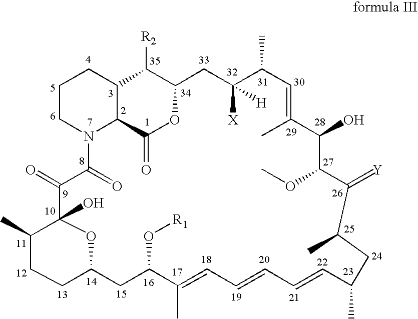

wherein R₁ is alkyl, alkenyl, alkynyl, hydroxyalkyl, hydroxyalkenyl, hydroxyalkynyl, benzyl, alkoxybenzyl or chlorobenzyl, R₂ is selected from formula IV or formula V:

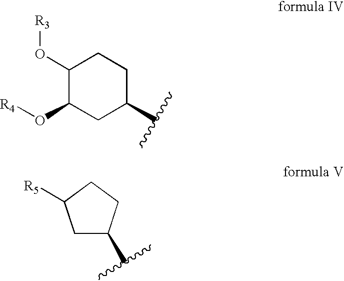

wherein R₃ is selected from H, alkyl, alkenyl, alkynyl, aryl, thioalkyl, arylalkyl, hydroxyarylalkyl, hydroxyaryl, hydroxyalkyl, dihydroxyalkyl, hydroxyalkoxyalkyl, hydroxyalkylarylalkyl, dihydroxyalkylarylalkyl, alkoxyalkyl, alkylcarbonyloxyalkyl, aminoalkyl, alkylaminoalkyl, alkoxycarbonylaminoalkyl, alkylcarbonylaminoalkyl, arylsulfonamidoalkyl, allyl, dihydroxyalkylallyl, dioxolanylallyl, carbalkoxyalkyl and alkylsilyl;

R₄ is H, methyl or together with R₃ forms C₂₋₆ alkylene;

R₅ is R₆O—CH₂—, wherein R₆ is selected from H, alkyl, alkenyl, alkynyl, aryl, alkylcarbonyl, arylcarbonyl, heteroarylcarbonyl, hydroxyalkylcarbonyl, aminoalkylcarbonyl, formyl, thioalkyl, arylalkyl, hydroxyarylalkyl, hydroxyaryl, hydroxyalkyl, dihydroxyalkyl, hydroxyalkoxyalkyl, hydroxyalkylarylalkyl, dihydroxyalkylarylalkyl, alkoxyalkyl, alkylcarbonyloxyalkyl, aminoalkyl, alkylaminoalkyl, alkoxycarbonylaminoalkyl, alkylcarbonylarninoalkyl, arylsulfonamidoalkyl, allyl, dihydroxyalkylallyl, dioxolanylallyl and carbalkoxyalkyl;

R₇CO—, wherein R₇ is selected from H, alkyl, hydroxy, alkoxy, aryloxy, amino, alkylamino, a residue of an amino acid, or N,N-disubstituted-amino wherein the substituents (a) are selected from alkyl, aryl or arylalkyl or (b) form a heterocyclic structure; R₈NCH—, wherein R₈ is alkyl, aryl, amino, alkylamino, arylamino, hydroxy, alkoxy or arylsulfonylamino; —O—CH—O—; or substituted dioxymethylyne;

Y is selected from O (H, OH), and (H, OR₉) wherein R₉ is selected from C₁₋₄ alkyl, alkylcarbonyl, arylcarbonyl, heteroarylcarbonyl, hydroxyalkylcarbonyl, aminoalkylcarbonyl, formyl or aryl; and

X is OH or H; wherein “alk” or “alkyl” refers to a C₁₋₁₀ aliphatic substituent optionally interrupted by an oxy linkage; and “ar” or “aryl” refers to a monocyclic, optionally heterocyclic, optionally substituted, C₄₋₁₄ aromatic substituent, provided that, when X is OH, R₁ is alkyl and R₂ is a residue of formula IV, then R₃ is other than H.

In the compounds of formula III, the following significances are preferred either individually or in any combination or sub-combination:
1. X is OH and R₁ is C₃₋₁₀-alkynyl or C₃₋₁₀ hydroxyalkynyl, preferably C₃₋₁₀ alk-2-ynyl or C₃₋₁₀ hydroxyalk-2-ynyl, more preferably C₃₋₆ alk-2-ynyl;
2. X is H and R, is C₁₋₁₀ alkyl, C₃₋₁₀ alk-2-enyl, C₃₋₁₀ hydroxyalk-2-enyl, C_{3-10 alk-}2-ynyl, C₃₋₁₀ hydroxyalk-2-ynyl or C₁₋₁₀ alkoxyC₁₋₁₀ alkyl, preferably C₁₋₆ alkyl or C₃₋₆ alk-2-ynyl, more preferably C₁₋₄ alkyl, most preferably methyl;
3. C₃₋₆ alkynyl as R₁ is 2-propynyl or pent-2-ynyl, preferably pent-2-ynyl;
4. Y is O, (H, OH) or (H, C₁₋₄ alkoxy), preferably O;
5. R₂ is a residue of formula IV;
6. In the residue of formula IV, R₃ is H, C₁₋₆ hydroxyalkyl, hydroxy-C₁₋₆ alkoxy-C₁₋₆ alkyl, (C₁₋₆ alkyl)-carbonyl-amino-C₁₋₆ alkyl, C₁₋₆ alkoxy-C₁₋₆ alkoxy or amino-C₁₋₆ alkyl, preferably H, hydroxyethyl, hydroxypropyl, hydroxyethoxyethyl, methoxyethyl or acetylaminoethyl; especially H when X is H or when X is OH and R₁ is alkynyl;
7. In the residue of formula IV, R₄ is methyl.
8. R₂ is a residue of formula V wherein R₅ is R₆ OCH₂— wherein R₆ is selected from H, C₁₋₆ alkyl, C₃₋₆ alk-2-enyl, C₃₋₆ alk-2-ynyl, aryl, C₁₋₆ alkyl-carbonyl, arylcarbonyl, hydroxyC₁₋₆ alkyl, C₁₋₆ alkoxy-C₁₋₆ alkyl or aminoC₁₋₆ alkyl; R₇ CO— wherein R₇ is selected from H, hydroxy, C₁₋₆ alkoxy, amino, C₁₋₆ alkylamino, a residue of an amino acid or N,N-disubstituted amino wherein the substituents (a) are selected from C₁₋₆ alkyl or aryl or (b) form a heterocyclic structure; R₈ NCH— wherein R₈ is alkyl, aryl, amino, alkylamino, arylamino, hydroxy, alkoxy or arylsulfonylamino; —O—CH—O—; or substituted dioxymethylyne.

Preferred compounds are compounds of formula IIIa:

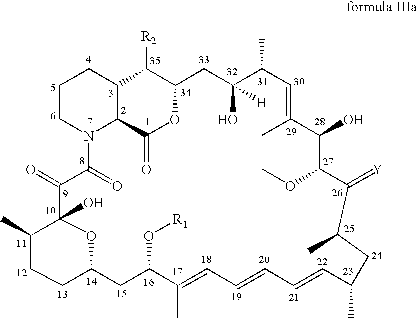

wherein R₁, R₂ and Y are as defined above, preferably have any of the preferred significances given under 1 and 3 to 8 above; and of formula IIIb:

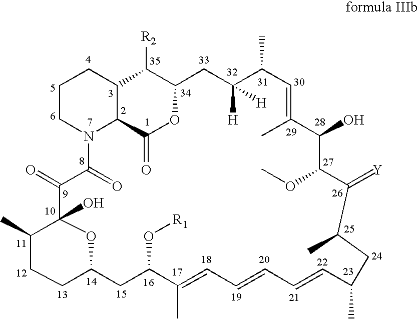

wherein R₁, R₂ and Y are as defined above, preferably have any of the preferred significances given under 2 to 8 above.

Especially preferred compounds include (i) 32-deoxo-rapamycin; (ii) 16-O-pent-2-ynyl-32-deoxo-rapamycin; (iii) 16-O-pent-2-ynyl-32-deoxo40-O-(2-hydroxy-ethyl)-rapamycin (iv) 16-O-pent-2-ynyl-32(S)-dihydro-rapamycin; (v) 16-O-pent-2-ynyl-32(S)-dihydro40-O-(2-hydroxyethyl)-rapamycin; (vi) 32(S)-dihydro40-O-(2-methoxytethyl-rapamycin; (vii) 32(S)-dihydro40-O-(2-hydroxyethyl)-rapamycin.

It has now surprisingly been found that compounds of formula I-III are useful for treating and/or preventing cardiovascular disorders. The compounds of the present invention are suitable as active agents in pharmaceutical compositions that are efficacious particularly for treating and/or preventing cardiovascular disorders. The pharmaceutical composition has a pharmaceutically effective amount of the present active agent along with other pharmaceutically acceptable excipients, carriers, fillers, diluents and the like. The term pharmaceutically effective amount as used herein indicates an amount necessary to administer to a host to achieve a therapeutic result.

The compounds described above are often used in the form of a pharmaceutically acceptable salt. Pharmaceutically acceptable salts include, when appropriate, pharmaceutically acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

As is evident to those skilled in the art, the many of the compounds of the present invention contain asymmetric carbon atoms. It should be understood, therefore, that the individual stereoisomers are contemplated as being included within the scope of this invention.

The compounds of the present invention can be produced by known organic synthesis methods as disclosed in U.S. Pat. No. 5,665,772, U.S. Pat. No. 5,352,671, U.S. Pat. No. 5,985,890, and U.S. Pat. No. 5,912,238.

The present invention further includes pharmaceutical compositions comprising a pharmaceutically effective amount of one or more of the above-described compounds as active ingredient. Pharmaceutical compositions according to the invention are suitable for enteral, such as oral or rectal, and parenteral administration to mammals, including man, for the treatment of pathological cardiac hypertrophy and heart failure, alone or in combination with one or more pharmaceutically acceptable carriers.

The compound is useful in the manufacture of pharmaceutical compositions having an effective amount the compound in conjunction or admixture with excipients or carriers suitable for either enteral or parenteral application. Preferred are tablets and gelatin capsules comprising the active ingredient together with (a) diluents; (b) lubricants, (c) binders (tablets); if desired, (d) disintegrants; and/or (e) absorbents, colorants, flavors and sweeteners. Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, the compositions may also contain other therapeutically valuable substances. The compositions are prepared according to conventional mixing, granulating or coating methods, respectively, and contain preferably about 1 to 50% of the active ingredient.

Suitable formulations also include formulations for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

In another embodiment, it is envisioned to use a compound of the present invention in combination with other therapeutic modalities. Thus, in addition to the therapies described above, one may also provide to the patient more “standard” pharmaceutical cardiac therapies. Examples of standard therapies include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 receptor antagonists and cytokine blockers/inhibitors.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, the compound therapy may precede or follow administration of the other agent by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

As discussed above, the compounds of the present invention are useful for treating and/or preventing cardiovascular disorders. Cardiovascular disorders as the term is used herein includes but is not limited to hypertension (whether of the malignant, essential, reno-vascular, diabetic, isolated systolic, or other secondary type), heart failure, such as congestive heart failure, angina (whether stable or unstable), myocardial infarction, atherosclerosis, diabetic nephropathy, diabetic cardiac myopathy pre and post-infarction, cardiac hypertrophy, cardiac fibrosis,renal insufficiency, peripheral vascular disease, left ventricular dysfunction, such as left ventricular hypertrophy, cognitive dysfunction (such as Alzheimer's, etc.), blood pressure-related cerebrovasular disease, stroke, pulmonary disease or pulmonary hypertension and headache, protection of end-organs, including the kidneys and the heart, for example protection against left ventricular hypertrophy, right ventricular hypertrophy, e.g. as associated with pulmonary hypertension, and the like, cardiomyopathy, vasculopathy and neuropathy and diseases of the coronary vessels.

The following examples are intended to illustrate the invention and are not to be construed as being limitations thereto.

### EXAMPLE 1

The ascending or transverse aortic-banded mouse models are used as pressure-overload models to ascertain the beneficial effects of the inventive agents (test agents) on pathological cardiac hypertrophy. The methods described by Tarnavski et al. (2004) or Ogita et al. (2004) are used for this purpose. Briefly, anesthetized C57BL/6 male mice (age, 11 to 12 weeks) are subjected to the surgical procedure of ascending or transverse aortic banding. Sham-operated mice are subjected to similar surgical procedures without constriction of the aorta.

Blood pressure and heart rate are measured non-invasively in conscious animals before and periodically after surgery by the tail-cuff plethysmography method. Under light anesthesia, 2-dimensional guided M-mode echocardiography is performed. The percentage of left ventricular fractional shortening is calculated as [(LVDD−LVSD)/LVDD]×100 (%) as described by Ogita et al. (2004). LVDD and LVSD indicate left ventricular end-diastolic and end-systolic chamber dimensions, respectively. Left ventricular mass was calculated as 1.055[(LVDD+PWTD+VSTD)3−(LVDD)3] (mg), where PWTD indicates diastolic posterior wall thickness, and VSTD indicates diastolic ventricular septal thickness.

After the above assessments, the animals are randomly segregated into aortic-banding or sham-operated groups. At the end of the aortic-banding operation, the animals are assigned to either the control (vehicle-treated) group or to the test (drug-treated) group. All groups are followed for not less than 4 weeks before using them for data analysis.

Hearts are excised after the mice are euthanized with an overdose injection of an anesthetic. Ratios of heart weight to body weight are ascertained. Sections of the hearts are prepared as previously described by Tarnavski et al. (2004), stained with hematoxylin-eosin and Masson's trichrome and observed under light microscopy.

### EXAMPLE 2

The beneficial effects of the inventive agents on cardiac hypertrophy are also ascertained in mice subjected to chronic infurion with an adrenoreceptor agonist. In these studies, male C57B1/6 mice (22-26 g) are surgically implanted with osmotic mini-pumps delivering isoproterenol (30 mg/kg/day) for periods not less than 14 days to induce cardiac hypertrophy. Control animals receive vehicle-loaded mini-pumps.

Blood pressure and heart rate are measured non-invasively in conscious animals before and periodically after surgery by the tail-cuff plethysmography method. Under light anesthesia, 2-dimensional guided M-mode echocardiography is performed. The percentage of left ventricular fractional shortening is calculated as [(LVDD−LVSD)/LVDD]×100 (%) as described by Ogita et al. (2004). LVDD and LVSD indicate left ventricular end-diastolic and end-systolic chamber dimensions, respectively. Left ventricular mass was calculated as 1.055[(LVDD+PWTD+VSTD)3−(LVDD)3] (mg), where PWTD indicates diastolic posterior wall thickness, and VSTD indicates diastolic ventricular septal thickness.

After the above assessments, the animals are randomly segregated into mini-pump implanted (vehicle/drug) or sham-operated groups. All groups are followed for not less than 14 days before using them for data analysis.

Hearts are excised after the mice are euthanized with an overdose injection of an anesthetic. Ratios of heart weight to body weight are ascertained. Transverse sections of the hearts are prepared as previously described by Tarnavski et al. (2004), stained with hematoxylin-eosin and Masson's trichrome and observed under light microscopy.

### EXAMPLE 3

The beneficial effects of the inventive compounds on cardiac hypertrophy and heart failure are ascertained in a murine model of myocardial infarction and heart failure. Myocardial infarction is induced in mice (age, 11-12 weeks) by ligating the left anterior descending (LAD) coronary artery under anesthesia as described by Tarnavski et al. (2004). Sham operated animals undergo the same experimental procedures but without coronary ligation.

Blood pressure and heart rate are measured non-invasively in conscious animals before and periodically after surgery by the tail-cuff plethysmography method. Under light anesthesia, 2-dimensional guided M-mode echocardiography is performed. The percentage of left ventricular fractional shortening is calculated as [(LVDD−LVSD)/LVDD]×100 (%) as described by Ogita et al. (2004). LVDD and LVSD indicate left ventricular end-diastolic and end-systolic chamber dimensions, respectively. Left ventricular mass was calculated as 1.055[(LVDD+PWTD+VSTD)3−(LVDD)3] (mg), where PWTD indicates diastolic posterior wall thickness, and VSTD indicates diastolic ventricular septal thickness.

An invasive method for blood pressure measurement is used prior to the animal sacrifice. A micromanometer tipped Millar catheter (1.4 French) is inserted into the right carotid artery and advanced into the LV chamber to measure LV pressure.

After the above assessments, the animals (ligated, sham operated) are segregated into 2 groups and treated with the inventive compounds or corresponding vehicles. All groups are followed for not less than 14 days before using them for data analysis.

Hearts are excised after the mice are euthanized with an overdose injection of an anesthetic. Ratios of heart weight to body weight are ascertained. Transverse sections of the hearts are prepared as previously described by Tarnavski et al. (2004), stained with hematoxylin-eosin and Masson's trichrome and observed under light microscopy.

### EXAMPLE 4

The beneficial effects of the inventive compounds on cardiac hypertrophy induced by tachycardia in dogs are also ascertained. The techniques described by Motte et al. (2003) with minor modifications are used in these studies. Briefly, a bipolar pacemaker lead is surgically advanced through the right jugular vein and implanted in the right ventricular apex of anesthetized mongrel dogs. A programmable pulse generator is inserted into a subcuticular cervical pocket and connected to the pacemaker lead.

The animals undergo a pacing protocol with a stepwise increase of stimulation frequencies as described by Motte et al. (2003). Pacing is initiated by activating the pulse generator at 180 beats/min and continued for 1 week, followed by 200 beats/min over a second week, 220 beats/min over a third week, and finally 240 beats/min over the last 2 wk. The investigations are carried out at baseline (week 0) and once weekly throughout the pacing period (i.e., from week 1 to week 5). On the third day of pacing, the test agent or matching placebo is administered and continued on the same daily dose until the end of the study at five weeks.

Body weight, rectal temperature, heart rate (HR), respiratory rate (RR), and blood pressure is monitored. Doppler echocardiography is performed under continuous ECG monitoring with a 3.57- to 5-MHz mechanical sector probe. Left ventricular internal end-diastolic (LVIDd) and systolic diameters (LVIDs) as well as systolic and diastolic left ventricular free wall (LVFWs and LVFWd) and interventricular septum thickness (IVSs and IVSd) are determined. An image of the aortic flow is obtained by pulsed-wave Doppler. The velocity spectra are used to measure the preejection period (PEP) and left ventricular ejection time (LVET). From these data, left ventricular end-diastolic (EDV) and systolic volume (ESV), left ventricular ejection fraction (LVEF), and mean velocity of circumferential fiber shortening (MVCF) are calculated.

Although the present invention has been described in considerable detail with reference to certain preferred versions thereof, other versions are possible without departing from the spirit and scope of the preferred versions contained herein.

All references referred to herein and set forth in the attached reference page, are hereby incorporated by reference in their entirety.

1. A method for treating and/or preventing pathologic cardiac disorders comprising administering to said mammal a compound of the formula (I) 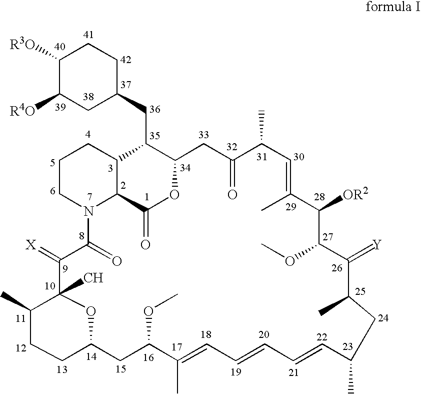
wherein
X is (H,H) or O;
Y is (H,OH) or O;
R¹ and R² are independently selected from
H, alkyl, arylalkyl, hydroxyalkyl, dihydroxyalkyl, hydroxyalkoxycarbonylalkyl, hydroxyalkylarylalkyl, dihydroxyalkylarylalkyl, acyloxyalkyl, aminoalkyl, alkylaminoalkyl, alkoxycarbonylaminoalkyl, acylaminoalkyl, arylsulfonamidoalkyl, allyl, dihydroxyalkylallyl, dioxolanylallyl, dialkyl-dioxolanylalkyl, di(alkoxycarbonyl)-triazolyl-alkyl and hydroxy-alkoxy-alkyl; wherein “alk-” or “alkyl” is C₁₋₆ alkyl, branched or linear; “aryl” is phenyl or tolyl; and acyl is a radical derived from a carboxylic acid; and
R⁴ is methyl or
R⁴ and R¹ together form C₂₋₆ alkyl;
provided that R¹ and R² are not both H; and hydroxyalkoxyalkyl is other than hydroxyalkoxymethyl. or a pharmaceutically acceptable salt thereof.

2. The method of claim 1 wherein the compound of formula I is 40-O-(2-hydroxy)ethyl-rapamycin.

3. A method for treating and/or preventing cardiovascular disorders comprising administering a therapeutically effective amount of 40-O-(2-hydroxy)ethyl-rapamycin to a subject in need thereof.

4. The method of claim 3 wherein the cardiovascular disorder is selected from the group consisting of hypertension, heart failure, angina, myocardial infarction, atherosclerosis, diabetic nephropathy, cardiac myopathy, cardiac hypertrophy, cardiac fibrosis, renal insufficiency, peripheral vascular disease, left ventricular dysfunction, cognitive dysfunction, blood pressure-related cerebrovasular disease, stroke, pulmonary disease, headache, end-organ damage, vasculopathy, neuropathy and diseases of the coronary vessels.

5. A method for treating and/or preventing pathologic cardiac disorders comprising administering to said mammal a compound of the formula (II) 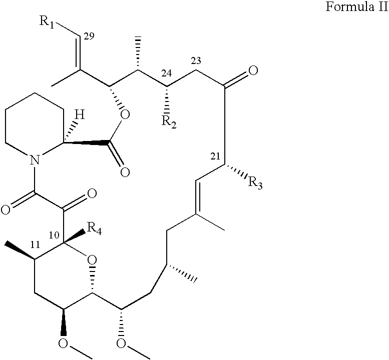
wherein R₁ is a group (a) of formula: 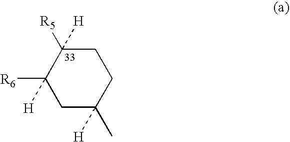
wherein R₅ is chloro, bromo, iodo or azido and R₆ is hydroxy or methoxy;
R₂ is oxo and there is a single bond in 23, 24 position; optionally protected hydroxy and there is a single or a double bond in 23, 24 position; or absent and there is a double bond in 23, 24 position; and
R₄ is hydroxy and there is a single bond in 10, 11 position; or absent and there is double bond in 10, 11 position;
R₃ is methyl, ethyl, n-propyl or allyl; in free form or, where such forms exist, in salt form.

6. The method of claim 5, wherein in the compound of formula (II), R₅ is chloro or bromo; R₆ is methoxy; and R₄ is hydroxy and there is a single bond in the 10, 11 position; R₂ is hydroxy or hydroxy protected by a hydroxy protecting group selected from formyl, tert-butoxycarbonyl, or tert-butyldimethylsilyl and there is a single or a double bond in the 23, 24 position; and R₃ is methyl, ethyl, n-propyl, or allyl, in free form.

7. The method of claim 5, wherein the compound of formula (II) is 33-epi-33-chloro-FR 520 (compound of Example 66a).

8. A method for treating and/or preventing pathologic cardiac disorders comprising administering to said mammal a compound of the formula (II) 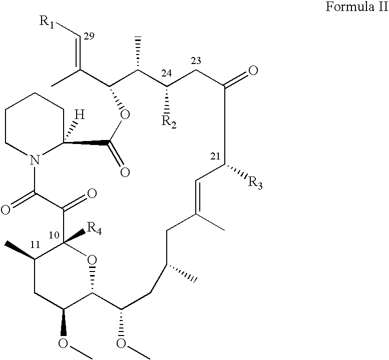
wherein R₁ is a group (b) or (d) of formula 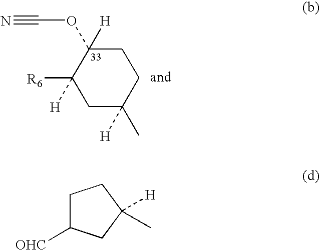
wherein R₆ is as defined above;
R₂ is as defined above; and
R₄ is hydroxyl and there is a single bond in 10, 11 position;
R₃ is methyl, ethyl, n-propyl or allyl; in free form or, where such forms exist, in salt form.

9. The method of claim 8, wherein in the compound of formula (II), R₁ is a group (b), R₆ is methoxy; R₂ is hydroxy protected by tert-butyldimethylsilyloxy and there is a single bond in 23, 24 position; or absent and there is a double bond in 23, 24 position; R₄ is hydroxy and there is a single bond in 10, 11 position; and R₃ is ethyl or allyl; in free form or, where such forms exist, in salt form.

10. The method of claim 8, wherein the compound of formula (II) is 29-des-(4-hydroxy-3-methoxycyclohexyl )-29-(3-formylcyclopentyl) -FR 520.

11. The method of claim 5, wherein the cardiovascular disorder is selected from the group consisting of hypertension, heart failure, angina, myocardial infarction, atherosclerosis, diabetic nephropathy, cardiac myopathy, cardiac hypertrophy, cardiac fibrosis, renal insufficiency, peripheral vascular disease, left ventricular dysfunction, cognitive dysfunction, blood pressure-related cerebrovasular disease, stroke, pulmonary disease, headache, end-organ damage, vasculopathy, neuropathy and diseases of the coronary vessels.

12. A method for treating and/or preventing pathologic cardiac disorders comprising administering to said mammal a compound of the formula (III) 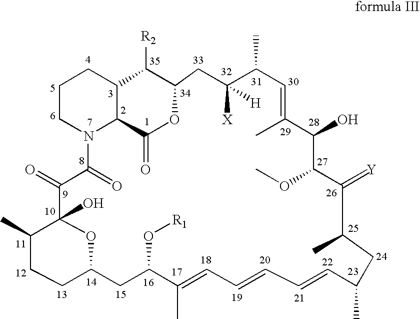
wherein R₁ is alkyl, alkenyl, alkynyl, hydroxyalkyl, hydroxyalkenyl, hydroxyalkynyl, benzyl, alkoxybenzyl or chlorobenzyl, R₂ is selected from formula IV or formula V: 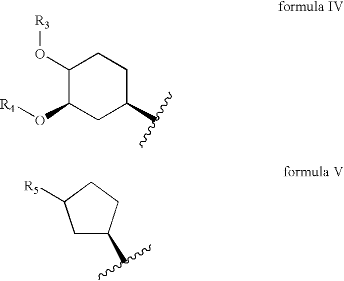
wherein R₃ is selected from H, alkyl, alkenyl, alkynyl, aryl, thioalkyl, arylalkyl, hydroxyarylalkyl, hydroxyaryl, hydroxyalkyl, dihydroxyalkyl, hydroxyalkoxyalkyl, hydroxyalkylarylalkyl, dihydroxyalkylarylalkyl, alkoxyalkyl, alkylcarbonyloxyalkyl, aminoalkyl, alkylaminoalkyl, alkoxycarbonylaminoalkyl, alkylcarbonylaminoalkyl, arylsulfonamidoalkyl, allyl, dihydroxyalkylallyl, dioxolanylallyl, carbalkoxyalkyl and alkylsilyl;
R₄ is H, methyl or together with R₃ forms C₂₋₆ alkylene;
R₅ is R₆O—CH₂—, wherein R₆ is selected from H, alkyl, alkenyl, alkynyl, aryl, alkylcarbonyl, arylcarbonyl, heteroarylcarbonyl, hydroxyalkylcarbonyl, aminoalkylcarbonyl, formyl, thioalkyl, arylalkyl, hydroxyarylalkyl, hydroxyaryl, hydroxyalkyl, dihydroxyalkyl, hydroxyalkoxyalkyl, hydroxyalkylarylalkyl, dihydroxyalkylarylalkyl, alkoxyalkyl, alkylcarbonyloxyalkyl, aminoalkyl, alkylaminoalkyl, alkoxycarbonylaminoalkyl, alkylcarbonylarninoalkyl, arylsulfonamidoalkyl, allyl, dihydroxyalkylallyl, dioxolanylallyl and carbalkoxyalkyl;
R₇CO—, wherein R₇ is selected from H, alkyl, hydroxy, alkoxy, aryloxy, amino, alkylamino, a residue of an amino acid, or N,N-disubstituted-amino wherein the substituents (a) are selected from alkyl, aryl or arylalkyl or (b) form a heterocyclic structure; R₈NCH—, wherein R₈ is alkyl, aryl, amino, alkylamino, arylamino, hydroxy, alkoxy or arylsulfonylamino; —O—CH—O—; or substituted dioxymethylyne;
Y is selected from O (H, OH), and (H, OR₉) wherein R₉ is selected from C₁₋₄ alkyl, alkylcarbonyl, arylcarbonyl, heteroarylcarbonyl, hydroxyalkylcarbonyl, aminoalkylcarbonyl, formyl or aryl; and
X is OH or H; wherein “alk” or “alkyl” refers to a C₁₋₁₀ aliphatic substituent optionally interrupted by an oxy linkage; and “ar” or “aryl” refers to a monocyclic, optionally heterocyclic, optionally substituted, C₄₋₁₄ aromatic substituent, provided that, when X is OH, R₁ is alkyl and R₂ is a residue of formula IV, then R₃ is other than H.

13. The method of claim 12, wherein the compound of formula (II) is 16-pent-2-ynyloxy-32(S)-dihydro-rapamycin or 16-pent-2-ynyloxy-32(S)-dihydro40-O-(2-hydroxyethyl)-rapamycin.

14. The method of claim 12, wherein the compound of formula (II) is 32-deoxo-rapamycin or 16-pent-2-ynyloxy-32-deoxo-rapamycin.

15. The method of claim 12, wherein the cardiovascular disorder is selected from the group consisting of hypertension, heart failure, angina, myocardial infarction, atherosclerosis, diabetic nephropathy, cardiac myopathy, cardiac hypertrophy, cardiac fibrosis, renal insufficiency, peripheral vascular disease, left ventricular dysfunction, cognitive dysfunction, blood pressure-related cerebrovasular disease, stroke, pulmonary disease, headache, end-organ damage, vasculopathy, neuropathy and diseases of the coronary vessels.

16. The method of claim 8, wherein the cardiovascular disorder is selected from the group consisting of hypertension, heart failure, angina, myocardial infarction, atherosclerosis, diabetic nephropathy, cardiac myopathy, cardiac hypertrophy, cardiac fibrosis, renal insufficiency, peripheral vascular disease, left ventricular dysfunction, cognitive dysfunction, blood pressure-related cerebrovasular disease, stroke, pulmonary disease, headache, end-organ damage, vasculopathy, neuropathy and diseases of the coronary vessels.
